(19) United States
(12) Patent Application Publication
(10) Pub. No.: US 2017/0119987 A1
(43) Pub. Date: May 4, 2017

(54) STRAPLESS NASAL INTERFACE DEVICE

(71) Applicant: Venkata Buddharaju, Park Ridge, IL (US)

(72) Inventor: Venkata Buddharaju, Park Ridge, IL (US)

(21) Appl. No.: 14/930,548

(22) Filed: Nov. 2, 2015

(51) Int. Cl. A61M 16/06; A61M 16/08; A61M 16/00

(52) U.S. Cl. CPC A61M 16/0666; A61M 16/0057; A61M 16/0605; A61M 16/0688; A61M 16/0816

(57) ABSTRACT

A breathing interface device includes a housing defining a chamber. The housing includes a first opening in fluid communication with the chamber and a positive pressure source, a first nostril tube in fluid communication with the chamber and having a first central axis, and a second nostril tube in fluid communication with the chamber and having a second central axis. The first nostril tube and the second nostril tube configured such that the first central axis and the second central axis of the second nostril tube intersect to form an angle. The breathing interface device further includes a nasal interface element that is removably couplable to the first nostril tube. The nasal interface element is compressible to fit within a nostril of a user and expandable to make contact with an inner edge of the nostril.

### FIELD OF THE INVENTION

The present invention relates to a strapless nasal interface device, and more particularly, to a device for use with a ventilator, CPAP, or BIPAP for strapless interface with the nasal passages.

### BACKGROUND

Nasal passage interface devices are commonly used in conjunction with CPAP devices, BIPAP, ventilators, and other devices. For example, in CPAP applications, a nasal passage interface device is used to provide a passageway for air to enter the nasal passages of the user. Such devices must be secured to the nasal passages so that it remains in place during sleep.

Various existing interfaces are typically held in place by straps or headgear. Such straps can become uncomfortable to the user, and can deter use of the device. Thus, existing devices are deficient in that they fail to provide an ideal method of attachment.

Numerous such interfaces have been disclosed. For example, U.S. Pat. No. 6,405,729, issued to Thornton, discloses an oral appliance for improving breathing. The appliance includes a mouthpiece and two nasal cushions which sit on the outside of the nose. The device requires the use of the mouthpiece to hold it in place, which would be uncomfortable for a user not in need of the mouthpiece. Without the mouthpiece, the nasal interface would not hold the unit in place.

U.S. Pat. No. 6,478,026, issued to Wood, discloses a nasal ventilation interface. The unit includes tubes which insert into the nose of the user. However, the inserts do not hold the unit in place. The devices require that the air tubes be looped over the ears of the user to hold the unit in place, which would be uncomfortable and easily dislodged during sleep.

Another device is disclosed in U.S. Pat. No. 6,581,594, issued to Drew et al. In this patent, a cumbersome respiratory mask is disclosed which covers the nose of the user. A forehead piece extends from the unit and is attachable to straps to hold the unit in place. The device is rather obtrusive and would have both straps to hold the device in place and a mask, both of which would be uncomfortable for a user.

Yet another device is disclosed in U.S. Pat. No. 7,000,613, issued to Wood et al. In this patent, an interface device also has tubes which extend into the nasal passage. Again, straps in the form of head straps are used to secure the device, which can cause discomfort to a user.

In U.S. Published Patent Application No. 2004/0182397, filed by Wood, a ventilation interface is disclosed having tubes extending into the nares of 5 the nose. In this disclosure, seal portions inserted into the nostrils are disclosed. However, these seal portions are not capable of securing the device to the user, and thus, further securing methods such as straps are needed, again causing potential discomfort to the user.

Another typical interface device is disclosed in U.S. Published Patent Application No. 2007/0272249, filed by Chandran et al. In this device, pillows are included which wedge into the nares of the nose. However, again, these pillows do not secure the device, and straps of various sorts would be required to hold it in place.

While a number of other interface devices have been used or disclosed, none of them enable securing via nasal inserts to eliminate the use of straps or air tube contraptions to secure the device to the user's nose. Furthermore, they each pose a level of potential discomfort to the user which render them problematic for sleeping.

Thus, there continues to be a need for a particular suitable nasal passage interface device which can be secured without the need for straps or air tube arrangements, and which provides a greater comfort level to the user.

### SUMMARY

The present invention provides, in one aspect, a strapless nasal interface device for use with a CPAP or BIPAP device, or any other ventilator type device. The nasal interface device of the present invention enables the user to secure the device in place without the need for straps or headgear, which can be uncomfortable for the user. Typical PAP devices are secured to the user via straps which wrap around the head of the user. These straps can be very uncomfortable, and can even deter use of the device altogether. Straps can be particularly uncomfortable when the head is tilted in various directions during sleep. This problem may also be particularly present in patients having claustrophobia issues when headgear or straps can be perceived as enclosing on a patient. Additionally, eliminating straps or headgear can make the maintenance and cleaning of the device easier. Providing a lightweight device which can be secured without straps or headgear is thus most advantageous.

The nasal interface device of the present invention includes an interface body which is preferably light weight and low profile. A ventilation tube is connected to the body, such as a tube through which air is supplied via a CPAP or other device. The body also includes exhalation holes. Thus, when the user exhales, the carbon dioxide rich exhalation air can exit through the exhalation holes, allowing new air to enter.

The body of the device interfaces with the nasal passage via nostril interface tubes extending from the body. These tubes include a nasal interface element designed to aid in securing the device in place, and thus eliminating the need for straps or headgear. The nasal interface elements are formed of an expandable, compressible material, meaning they can be compressed in order to insert them into the nostril, and once inserted they expand to apply pressure to the inner surfaces of the nostril opening to hold the device in place. Preferably, the contact is made at the end portion of the nostril so that only the section in the nostril covered by skin is contacted, thus avoiding the more sensitive nasal mucosa. Alternatively, the nostril interface elements are held against the outer edge of the nostrils.

These nasal interface elements can be formed of any suitable material which can be deformed, but are resilient in that they tend back to their original shape. A compressible foam is one material which may be well-suited for use in the invention. The interface element can be either permanently mounted to the interface tubes, or they may be removable and disposable so that they can be replaced after use. Thus, they can be mounted to the tubes via any suitable method. For example, they can be mounted via threading, frictional fit, or adhesive. However, other suitable methods of mounting the element to the tubes are contemplated and considered within the scope of the present invention.

The nasal interface elements of the present invention are ideally shaped in such a manner that they will form a seal or snug fit within the nostril of the user. This may mean they have more material on the bottom part toward the user's face, or that they may be specially formed to conform to the shape of the interior of the nostril. By exerting a small amount of pressure to the inside of the nostril, an additional advantage can be obtained by widening the passageway to allow more airflow. Furthermore, to enhance the securing of the device, the interface elements may include a mild adhesive on their exterior surface to hold them in place against the inner surfaces of the nostril. Such adhesive must be mild enough that the element can be easily removed by the user without causing significant discomfort or irritation or abrasion.

Optionally, these interface elements may contain an anti-bacterial agent to reduce the growth of contaminants. Once used, the user may replace the elements onto the tubes.

In various embodiments of the present invention, the interface tubes may further include a skirt surrounding the tube below the interface elements. Such skirts can be fitted against the outside of the nostril opening to help form a seal. Optionally, a mild adhesive can be disposed on the surface of the skirt which touches the outside edge of the nasal opening to help hold it in place.

In various embodiments of the present invention, the device also includes one or more flaps to assist in holding the device in place while in use. For example, side flaps are optionally included extending from the sides of the body of the device. The side flaps have an adhesive, enabling them to be adhered to the outer surface of the nose of the user to secure the device in place. Alternatively, the side flaps may removably secure to a nose cover which is adhered to the nose of the user.

Furthermore, an optional front flap may be included. The front flap extends from the body of the device and is secured to the exterior surface of the nose of the user. It may be secured via a strip of tape, and adhesive on the underside of the flap, or by any other suitable method. This front flap holds the device in place during use. In certain embodiments, the front flap may include resilient strips traversing the exterior of the nasal passageways. These strips can be deformed when applying the front flap to the nose via an adhesive, and will apply pressure tending to open the nasal passageways. A single one of such strips can be used. Alternatively, multiple strips can be used. Thus, the passageway is opened to allow more airflow, reducing the tendency of snoring in the user, and potentially reducing the air pressure required through the device to accomplish its purpose. Greater airflow may be enhanced by the combination of the strips and the expanding nasal interface elements, thus reducing air pressure requirements.

Still other objects and advantages of the present invention will become readily apparent to those skilled in the art from the following detailed description, wherein it is shown and described only the preferred embodiments of the invention, simply by way of illustration of the best mode contemplated of carrying out the invention. As will be realized, the invention is capable of other and different embodiments and its several details are capable of modifications in various obvious respects, without departing from the invention. Accordingly, the drawings, wherein like reference numerals represent like features, and description are to be regarded as illustrative in nature and not as restrictive.

In another aspect, a breathing interface device includes a housing defining a chamber. The housing includes a first opening in fluid communication with the chamber and a positive pressure source. The housing further includes a first nostril tube in fluid communication with the chamber and a second nostril tube in fluid communication with the chamber. The first nostril tube has a first central axis and the second nostril tube has a second central axis. The first nostril tube and the second nostril tube are configured such that the first central axis and the second central axis of the second nostril tube intersect to form an angle. The breathing interface device further includes a nasal interface element removably couplable to the first nostril tube. The nasal interface element is compressible to fit within a nostril of a user and expandable to make contact with an inner edge of the nostril.

In yet another aspect, a breathing interface device includes a housing defining a chamber. The housing includes a first nostril tube in fluid communication with the chamber, a second nostril tube in fluid communication with the chamber, and a first opening in fluid communication with the chamber and a positive pressure source. The breathing interface device further includes a facial patch adherable to a portion of a user's face and has a contact surface, and an attachment flap rotatably couplable to the housing and releasably couplable to the facial patch. The breathing interface device further includes a nasal interface element removably couplable to the first nostril tube. The nasal interface element is compressible to fit within a nostril of a user and expandable to make contact with an inner edge of the nostril.

In still yet another aspect, a breathing interface device includes a housing defining a chamber. The housing includes a first opening in fluid communication with the chamber and a positive pressure source. The first opening has a central opening axis. The housing further includes a first nostril tube in fluid communication with the chamber and a second nostril tube in fluid communication with the chamber. The housing further includes a wall having a surface angled relative to the central opening axis of the first opening. The first nostril tube extends from the surface. The breathing interface defines further includes a nasal interface element removably couplable to the first nostril tube. The nasal interface element is compressible to fit within a nostril of a user and expandable to make contact with an inner edge of the nostril.

### BRIEF DESCRIPTION OF THE DRAWINGS

FIG. 1 shows an embodiment of the nasal interface device of the present invention.

FIG. 2 shows another embodiment of the nasal interface device of the present invention, having optional side and front straps for mounting the device.

FIG. 3 shows an embodiment of the nasal interface device of the present invention as mounted on the nose of a user.

FIG. 4 shows an embodiment of the nasal interface element of the present invention.

FIG. 5 shows another embodiment of the nasal interface device of the present invention.

FIG. 6 shows an exploded view of one embodiment of the nasal interface device of the present invention.

FIG. 7 shows a perspective view of one embodiment of the nasal interface device of the present invention.

FIG. 8 shows another embodiment of the nasal interface device of the present invention.

FIG. 9 shows a front perspective view of another embodiment of a nasal interface device.

FIG. 10 shows an exploded front perspective view of the nasal interface device of FIG. 9.

FIG. 11 shows a rear perspective view of the nasal interface device of FIG. 9.

FIG. 12 shows an exploded rear perspective view of the nasal interface device of FIG. 9.

FIG. 13 shows a front view of the nasal interface device of FIG. 9.

FIG. 14 shows a side view of the nasal interface device of FIG. 9.

FIG. 15 shows a top view of the nasal interface device of FIG. 9.

FIG. 16 shows a front perspective view of a nasal interface body of the nasal interface device of FIG. 9.

FIG. 17 shows a bottom perspective view of the nasal interface body of FIG. 16.

FIG. 18 shows a side view of the nasal interface body of FIG. 16.

FIG. 19 shows a cross-sectional view of the nasal interface device of FIG. 9 taken along line 19-19 in FIG. 13.

FIG. 20 shows a front view of the nasal interface device of FIG. 9, illustrating the use of attachment flaps.

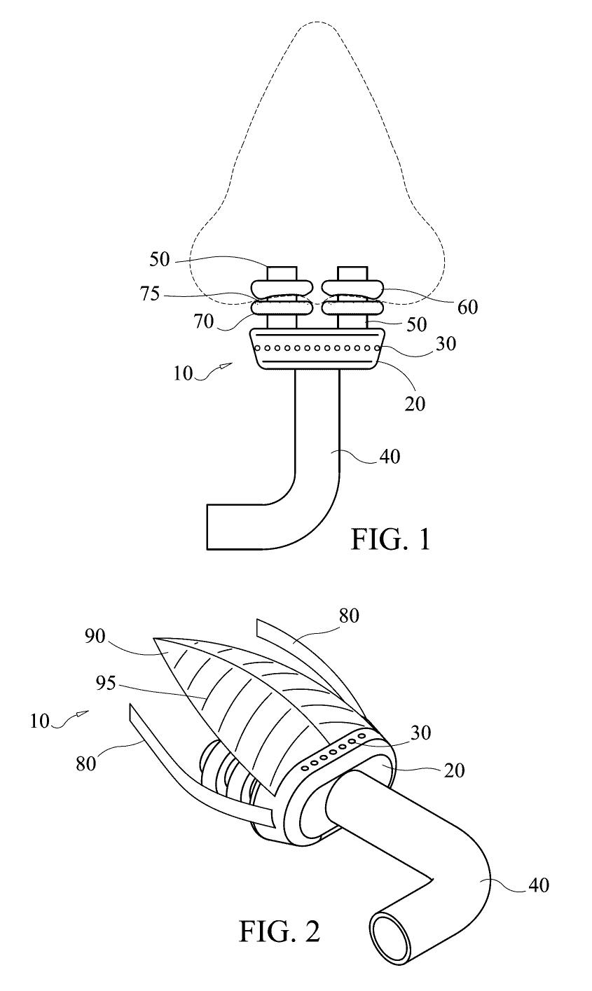

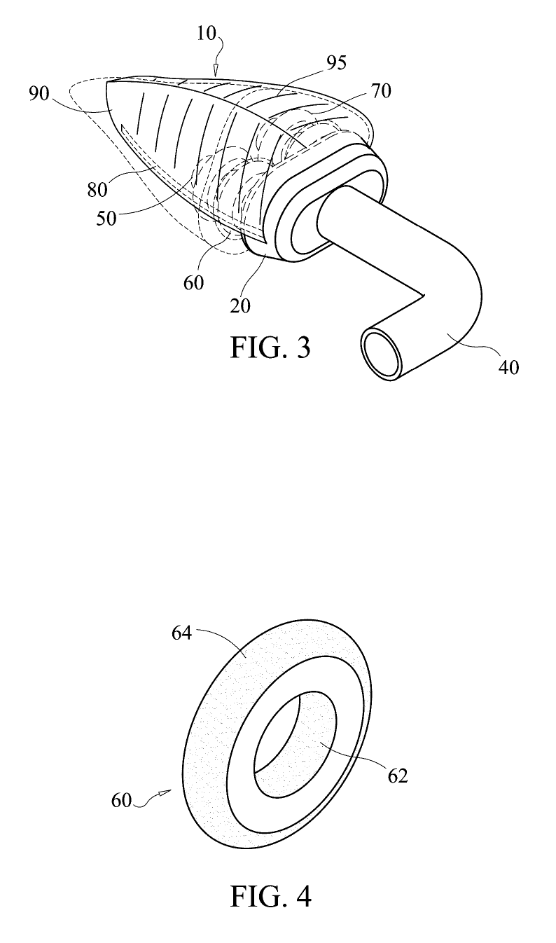

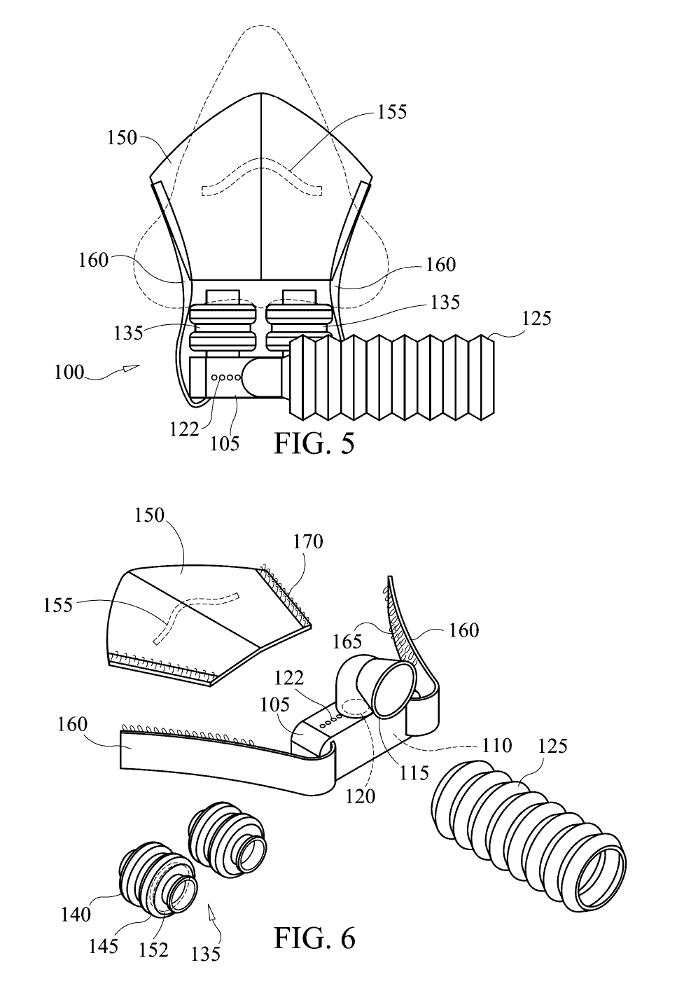

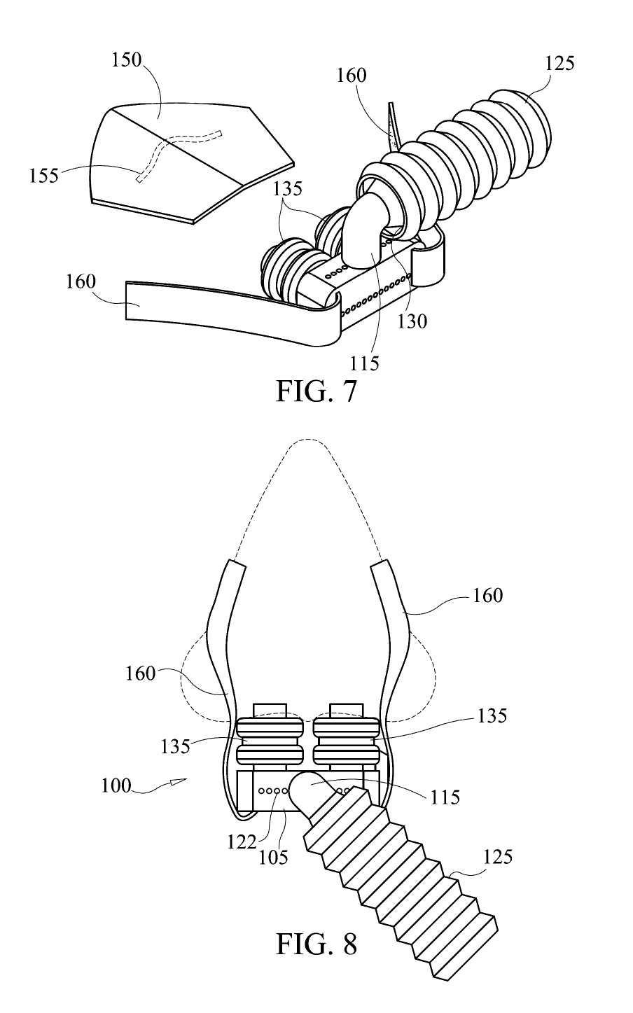

### DETAILED DESCRIPTION OF THE INVENTION

While this invention is susceptible of embodiments in many different forms, there are shown in the drawings and will herein be described in detail, preferred embodiments of the invention with the understanding that the present disclosure is to be considered as an exemplification of the principles of the invention and is not intended to limit the broad aspect of the invention to the embodiments illustrated.

The present invention is a strapless nasal interface device 10 suitable for use with a CPAP or BIPAP device, or any other ventilator type device. The device 10 includes an interface body 20 having an interior cavity. The cavity is fluidly connected to an air tube 40 extending from the body 20 for use with a CPAP or other device. The body 20 ideally includes exhalation holes 30 to enable expelled air to escape the device 10.

The device 10 further includes at least one nostril interface tube 50 extending from the body 20 to interface with the nasal passages of the user. Preferably, two such tubes 50 are included. Surrounding the tubes are compressible expandable nasal interface elements 60. These elements 60 are formed such that they can be compressed to fit within the nostril of the user, after which they expand to make contact with the interior of the nostril at the inner edge of the nostril to hold the interface tube 50 in place within the nostril. Thus, the need for straps or headgear in conjunction with the device is eliminated.

In various embodiments of the present invention, the interface elements 60 are permanently mounted to the interface tubes 50. However, in various other embodiments, the interface elements 60 are removably mounted to the tubes 50. If removably mounted, the interface elements 60 may be mounted to the tubes 50 by any suitable means. For example, they may be threaded, frictionally fit, or adhered with an adhesive 62.

Interface elements 60 can be formed of any suitable material. In certain embodiments, they are formed of an expandable, compressible foam. Preferably, the elements 60 have an anti-bacterial agent. Optionally, elements 60 may include a mild adhesive 64 on the outer surface to make contact with the skin on the interior of the nostril. Ideally, elements 60 form a seal within the nostril. Ideally, they are shaped to optimize such a seal. For example, they may be formed to conform to the interior shape of the nostril so as to form a proper fit.

In various embodiments of the present invention, a skirt 70 is also included. The skirt 70 is disposed on the nostril interface tube 50 between the interface element 60 and the interface body 20. The skirt interfaces with the end of the nostril opening to help aid in sealing the air passage. Optionally, a mild adhesive can be disposed on the surface 75 of the skirt 70 adjacent the rim of the nostril opening to further seal the passageway.

In various further embodiments, the device 10 includes one or more flaps in order to further secure it on the user. For example, in certain embodiments, side flaps 80 extend from the body 20. The side flaps 80 have an adhesive thereon to adhere the flaps to the exterior of the nose, further securing it. Side flaps 80 may be mounted to the interface body 20 by any suitable method, such as via a groove in the interface body 20 allowing an end of flap to be inserted. Such a fit may be a friction fit, or it may be a dovetail groove. However, any other suitable method of attachment is contemplated as within the scope of the invention.

Additionally, an optional front flap 90 may extend from the interface body 20. The front flap 90 covers an exterior portion of the nose. It can be mounted to the nose via any suitable method. For example, a separate strip of tape or adhesive may be used. In certain embodiments, it may be mounted via an adhesive on the underside of the front flap 90. Thus, the device 10 is further secured to the nose of the user. Front flap 90 may be mounted to the interface body 20 by any suitable method, such as via a groove in the interface body 20 allowing an end of flap 90 to be inserted. Such a fit may be a friction fit, or it may be a dovetail groove. However, any other suitable method of attachment is contemplated as within the scope of the invention.

In various embodiments, the front flap 90 of the device 10 further includes tension bows or strips 95. These strips 95 are formed of a resilient material. Thus, the strips 95 may be deformed when adhered to the exterior of the nose via an adhesive on the underside of the front flap 90, after which they apply pressure tending to expand the nasal passageway. Thus, the passageways are expanded, relieving a tendency of snoring and potentially reducing the air pressure required for the user's purposes. While a single such strip 95 can be used, in certain embodiments multiple strips 95 are used to open the passageway along an entire portion of the passageway. The front flap 90 can be used without side flaps 80, or in conjunction therewith.

In various other embodiments, the present invention includes a strapless nasal interface device 100 having an interface body 105 having an internal cavity 110. The cavity 110 is in communication with an air tube 115 which extends from the body 105. The air tube 115 is preferably rotatably attached to the interface body 105 at a first point of attachment 120. Ideally, this point of attachment 120 allows the air tube 115 to rotate 360 degrees, yet maintains a substantially leak-proof connection regardless of how the air tube 115 is oriented with respect to the interface body 105. A sealing o-ring may be incorporated to facilitate the rotatable mounting. This first point of attachment 120 may be located at any suitable position on interface body 105, however, in a preferred embodiment it is located on a front wall which is adjacent the wall on which nostril interface tubes 135 are located such that the air tube 115 extends out perpendicular to the plane of the face and away from the face, thus keeping air tube 115 and air supply tube 125 from rubbing against the face of the user.

Preferably, the device 100 comprises at least one exhalation hole, and preferably a plurality of exhalation ports or holes 122 which are sized to allow exhaled air to pass therethrough, but which are sized so that they do not substantially depressurize the cavity 110 of the interface body 105. Exhalation holes 122 can be placed in any suitable location on the interface body 105. For example, they may be placed on the side opposite of where nostril interface tubes 135 are located, or on the top side of the interface body 105 near the first point of attachment 120, or in both locations, or in any other suitable location. In certain embodiments, 35 to 40 exhalation holes sized at about 0.023 inches each may be suitable to enable carbon dioxide rich exhalation air to leave the cavity 110, yet such holes 122 are small enough such that air pressure intended to pressurize the nasal passage is maintained. In another embodiment, hole sizes may be approximately 0.02 inches in diameter, and about 78 of them may be suitable. However, any size, number, and configuration of exhalation holes 122 which function as described is suitable and contemplated within the scope of the present invention.

In various embodiments, the air tube 115 is also attached to an air supply tube 125. Preferably, this is a removable attachment. Optionally, the connection at a second attachment point 130 between the air tube 115 and the air supply tube 125 also allows rotation while maintaining a leak-proof connection. Ideally, 360 degrees of rotation is enabled. In certain embodiments, the air tube 115 is bent somewhere along its length, such that the planes of rotation at the two attachment points 120 and 130 are different planes of rotation. For example, if the air tube is bent by about 90 degrees, the planes of rotation will be substantially orthogonal with respect to one another. In some embodiments, one plane of rotation will be parallel to the face of the user, and the other will be perpendicular to the user. This allows for a great degree of freedom of movement of the user without compromising the integrity of the seal formed between the device and the user's nostrils.

In various embodiments, the device includes at least one nostril interface tube 135 having an internal passage extending from the interface body 105 to the nostrils of a user. Preferably, there are two such tubes or nostril interface elements 135 extending to the nostrils of a user. In certain embodiments, the nostril interface elements 135 include an upper cushion 140, which is a radial protrusion, preferably formed of compressible material such that the upper surface of the cushion can be compressed against the outer edge of the nostril to help form a seal with the nostril of the user. Ideally, nostril interface tubes 135 are each formed as a single integral piece which can be attached and removed from the interface body 105.

Preferably, the nostril interface elements 135 also include a lower radial element 145 which is a radial protrusion adapted to be urged toward the upper cushion 140 and to compress the upper cushion 140 to aid in forming a seal between the upper cushion 140 and a person's nostril when the device 100 is worn by a person. In certain embodiments, the lower radial element 145 includes an internal substantially rigid rim 152 adapted to aid in compressing the upper cushion 140 when the device 100 is worn by a person.

Ideally, the nostril interface elements 135 have substantially non-porous surfaces. For example, they may be formed entirely of a non-porous material such as silicone or any other suitable material. Alternatively, at least the exposed surfaces of the nostril interface elements 135 are coated with a non-porous material such as silicone, or any other suitable material. In certain embodiments, the nostril interface elements 135 are removably attached to the interface body 105, such as via a snap fit, frictional fit, threading, or any other suitable method. Thus, they can be removed for cleaning or replacement.

The device 100 includes a facial patch 150. The facial patch can be adhered to the face of a user. Preferably, the facial patch 150 is a nose cover 150 which adheres to the nose of a user. The nose cover 150 preferably includes an adhesive on its bottom surface to removably adhere to the skin of the user's nose. The adhesive is ideally suitable for use on skin and removable. This nose cover 150 is ideally disposable and replaceable.

The nose cover 150 is ideally formed of a flexible material. Optionally, a bendable element 155 is included as part of the nose cover 150 which may be embedded within it or on one of its surfaces. The bendable element 155 is formed of a material which can be bent to fit the contour of the nose and hold its shape so as to help the nose cover 150 adhere to the nose. The bendable element may be formed of any suitable material, such as a metal as aluminum, or any other material with suitable properties.

In various embodiments, in order to hold the device 100 in place properly, at least one attachment flap 160, and preferably two attachment flaps 160 are connected to the nasal interface body 105. They secure to the surface of the nose cover 150. Any suitable manner of removable attachment is contemplated. For example, hook and loop attachment in which one element of the hook and loop material 165 is attached to the flaps 160, and the other element of the hook and loop material 170 is attached to the surface of the nose cover 150. However, an adhesive or other manner of attachment can be used. Ideally, the flaps 160 extend around the sides of the interface body 105 and hold the device 100 snug against the nostrils. In this way, no straps around the head of the user are required.

In an alternate embodiment, the nostril interface elements 135 may include a magnet or iron or nickel bearing material, and they may be secured to the nose via magnets on the external surface of the nose. In a further alternate embodiment, as shown in FIG. 8, the nose cover 150 may be eliminated and one or more flaps 160 may be adhered directly to the outer surface of the nose of the user.

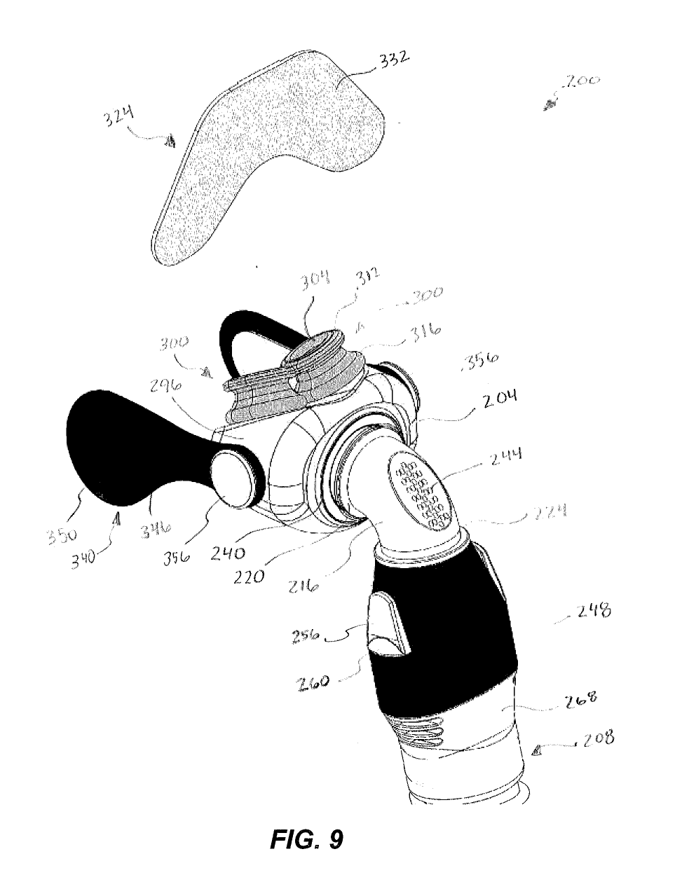

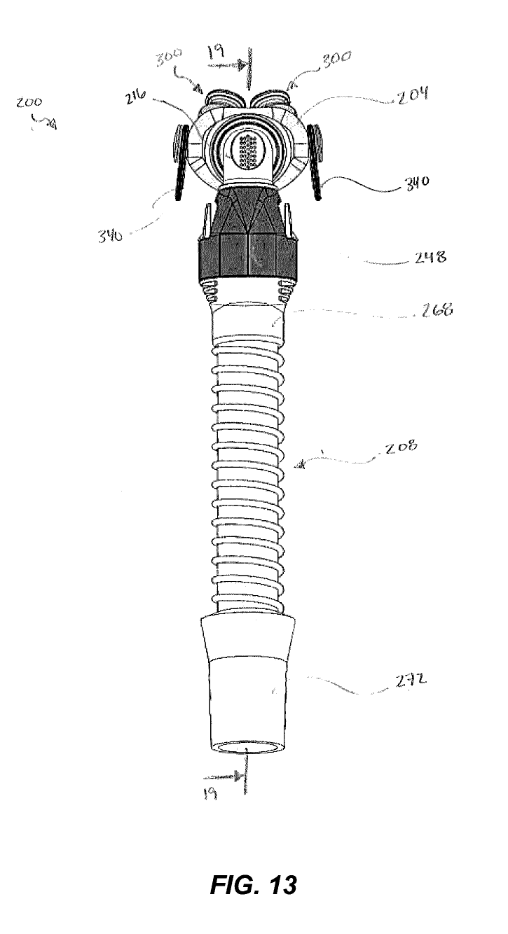

FIGS. 9-15 and 19-20 illustrate a strapless nasal interface device 200 according to another embodiment and suitable for use with a CPAP or BIPAP device, or with any other ventilator type device. The device 200 includes an interface body 204 defining an interior cavity or chamber 206, an intermediate air tube 216, and an air supply tube 208 extending therefrom.

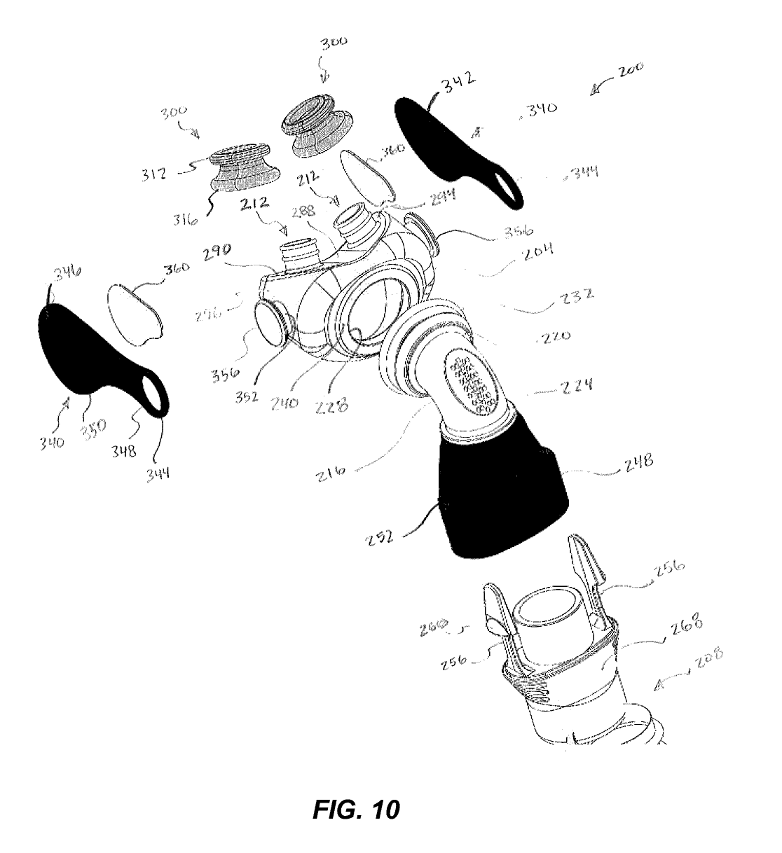

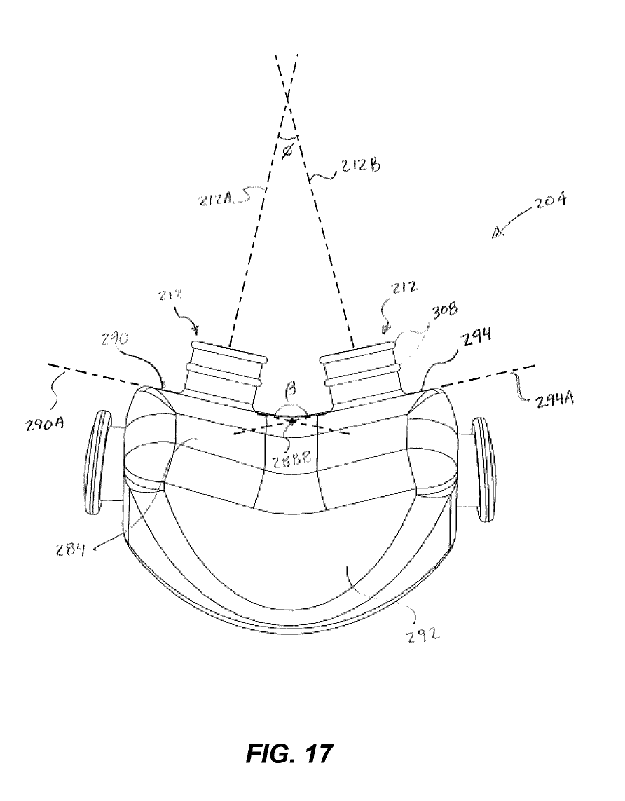

With reference to FIGS. 10, 12, and 16-18, the nasal interface body 204 includes a housing having a first wall 284, a second wall 288, a third wall 292, and a pair of side walls 296. The body 204 further includes a generally arcuate wall 240, which defines an opening 228 opposite the first wall 284 and having a central axis 228A.

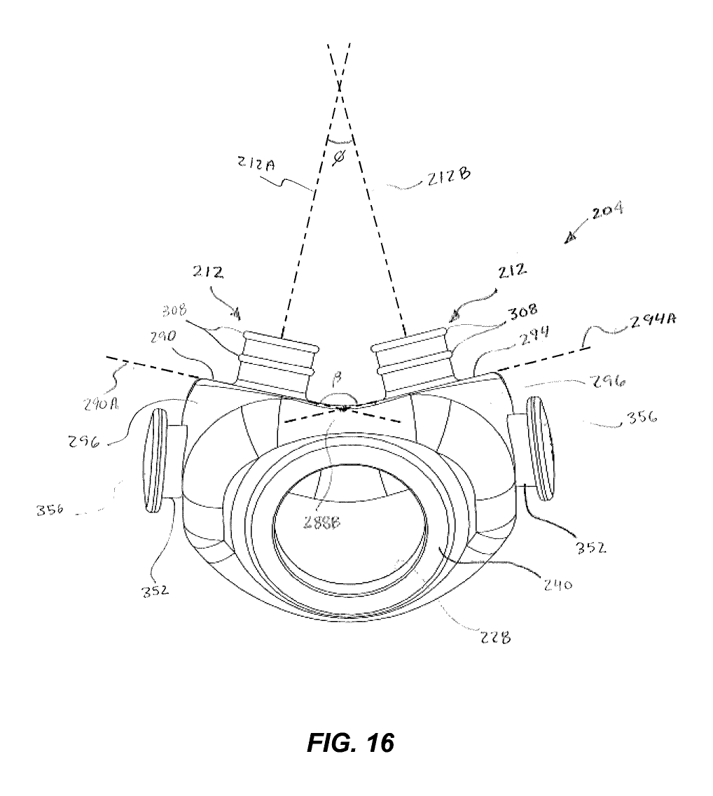

Referring to FIGS. 16-18, the second wall 288 comprises first and second surfaces 290, 294 coincident with respective planes 290A, 294A. The planes 290A, 294A intersect along a line of intersection 288B and are angled relative to each other such that the planes 290A, 294A form an obtuse angle β of between about 140 degrees and about 170 degrees, and preferably of about 155 degrees.

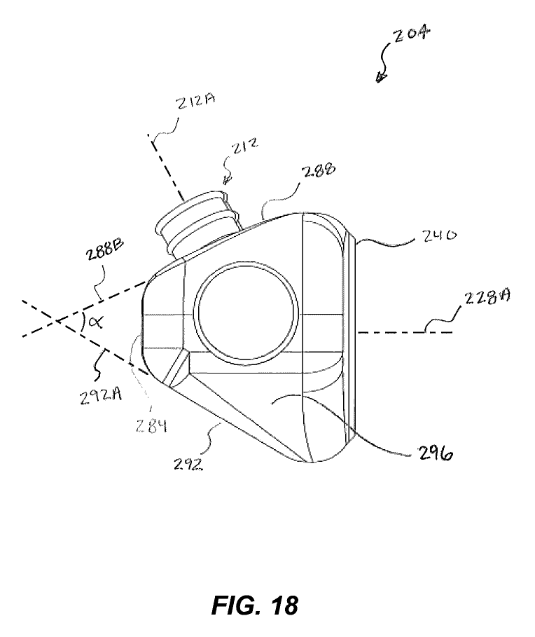

The third wall 292 surface is coincident with a plane 292A, which intersects the line of intersection 288B (in the view of FIG. 18) to form an acute angle α between about 40 degrees and about 80 degrees, and preferably of about 60 degrees. The third wall 292 and the second wall 288 are therefore angled relative to the central axis 228A of the primary opening 228 of the body 204.

A pair of nasal interface tubes 212 extend from the body 204 and in particular from an associated plane 290A, 294A of the second wall 288. Each of the nasal interface tubes 212 is in fluid communication with the cavity 206 of the body 204 and has a central longitudinal axis 212A, 212B normal to the respective plane 290A, 294A. As such, the central axes 212A, 212B of the nasal interface tubes 212 intersect to form an acute angle φ that is at least about 20 degrees, and is preferably between about 20 degrees and about 40 degrees, and is more preferably about 27 degrees. The nasal interface tubes 212 are consequently tilted with the planes 290A, 294A and extend normally therefrom. The nasal interface tubes 212 further include annular ribs 308 on an exterior surface and concentric with the respective axes 212A, 212B. The ribs 308 may be integrally formed as one piece with each tube 212 or otherwise fixedly attached to each tube 212.

An extension 352 protruding from each side wall 296 of the body 204 presents a flange portion 356, the purpose of which will be further detailed below. The entirety of the body 204 is preferably formed from plastic and is, for example, molded, plastically welded, or similarly constructed.

Referring also to FIGS. 9-12, the device 200 further includes flexible nasal interface elements, pillows, buds, or cushions 300 formed from expandable, compressible foam, but alternatively may be constructed from any suitable material. Each of the generally cylindrical nasal interface elements 300 defines a passage 304 therethrough such that each element 300 is configured to be axially fitted over a respective nasal interface tube 212 and held in place on the tube 212. The nasal interface elements 300 are preferably removable, disposable, and replaceable. In alternative embodiments, the nasal interface elements 300 are fixedly secured to the tubes 212 by adhesive or another suitable method.

The outer contours of the nasal interface elements 300 are shaped to form a seal within the user's nostrils. In particular, each of the nasal interface elements 300 includes an upper radial element 312 and a lower radial element 316. The upper radial element 312 is compressible to fit within one of the nostrils of the user.

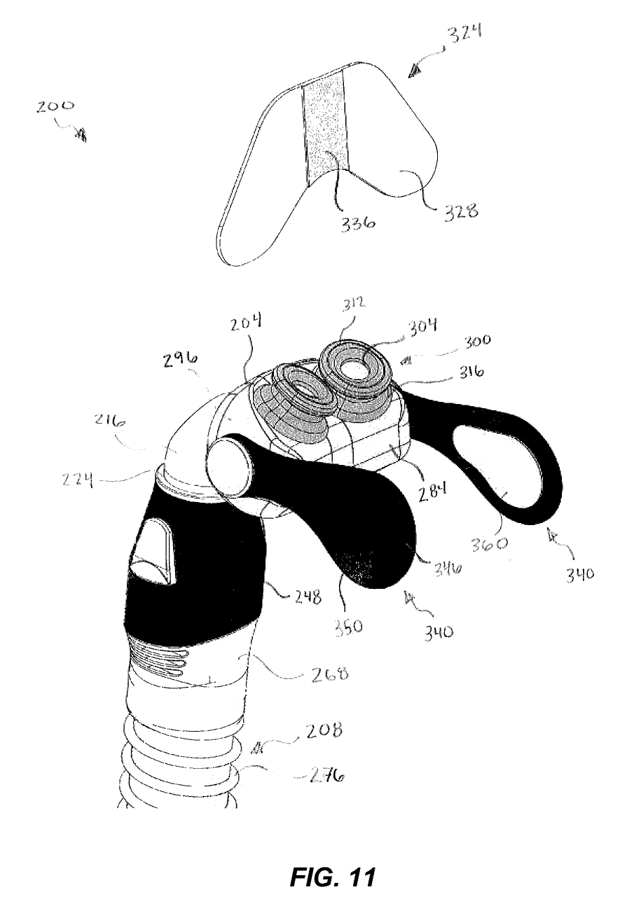

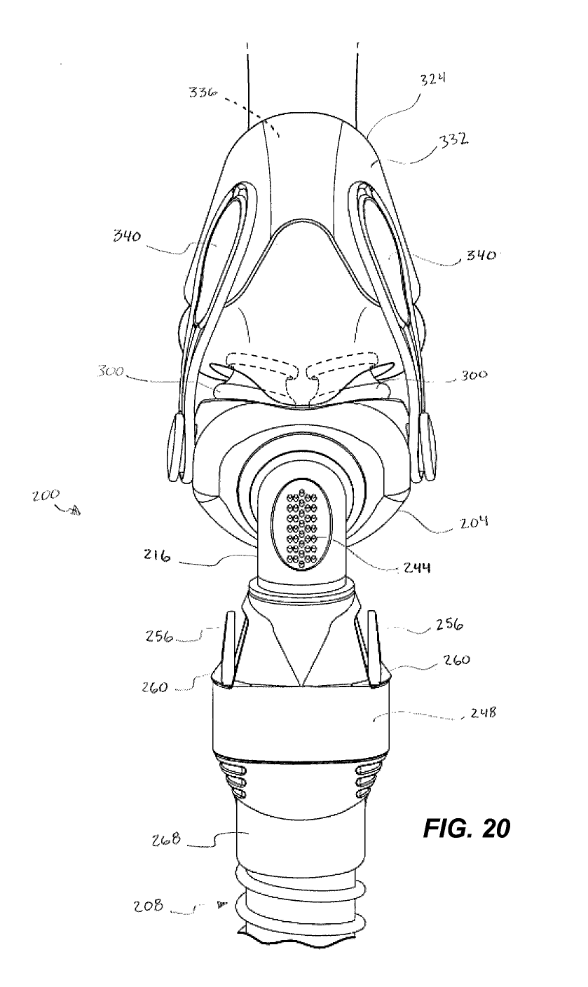

With reference to FIGS. 9, 11, and 20, the device 200 further includes a facial patch 324 configured to be adhered to a portion of the face of the user. In the illustrated embodiment, the facial patch 324 is in the form of a nose cover removably attachable to a user's nose. The nose cover 324 is formed of a flexible material and includes a front surface 332 (FIG. 9) to facilitate manipulation of the nose cover 324 and a contact surface 328 (FIG. 11). All or a portion of the contact surface 328 includes adhesive, which is ideally suitable for use on skin and removable therefrom. In addition, a centrally located masked area 336 extends along a centerline of the back surface 328 with a width that generally corresponds to and overlays the bridge of the nose of the user, as shown in FIG. 20. The masked area 336 is free of adhesive. The width of the central masked area 336 is between about 1.0 cm and about 1.5 cm (e.g., about 1.25 cm). The nose cover 324 is preferably disposable and replaceable.

In the illustrated embodiment, the device 200 further includes a pair of flexible attachment flaps 340 connectable to the interface body 204 and configured to hold the device 200 in position. Each of the attachment flaps 340 includes a ring portion 344 configured to receive a corresponding extension 352 and flange portion 356 for pivotal coupling thereto. Each of the flaps 340 further includes reinforcement ribs 346, 348, 350. In particular, a reinforcement rib 346 extends around or near all or a subsection of the periphery of a contact portion 342 of each of the flaps 340, and a reinforcement rib 348 also extends around or near all or a subsection of the periphery of the ring portion 344. An additional reinforcement rib 350 may be offset from the reinforcement rib 346 around at least a portion of the perimeter of the flap 340.

With reference to FIG. 20, the contact portion 342 of the each attachment flap 340 is configured to be secured to the adjacent front surface 332 of the nose cover 324. Any suitable manner of removable attachment is contemplated. As one example, hook and loop fastening may be used, in which one element of hook and loop material 360 is affixed to the attachment flaps 340 and the other element of hook and loop material is affixed to the front surface of the nose cover 324. In other embodiments, an adhesive, a magnet, or other manner of attachment may be used in lieu of hook and loop fastening.

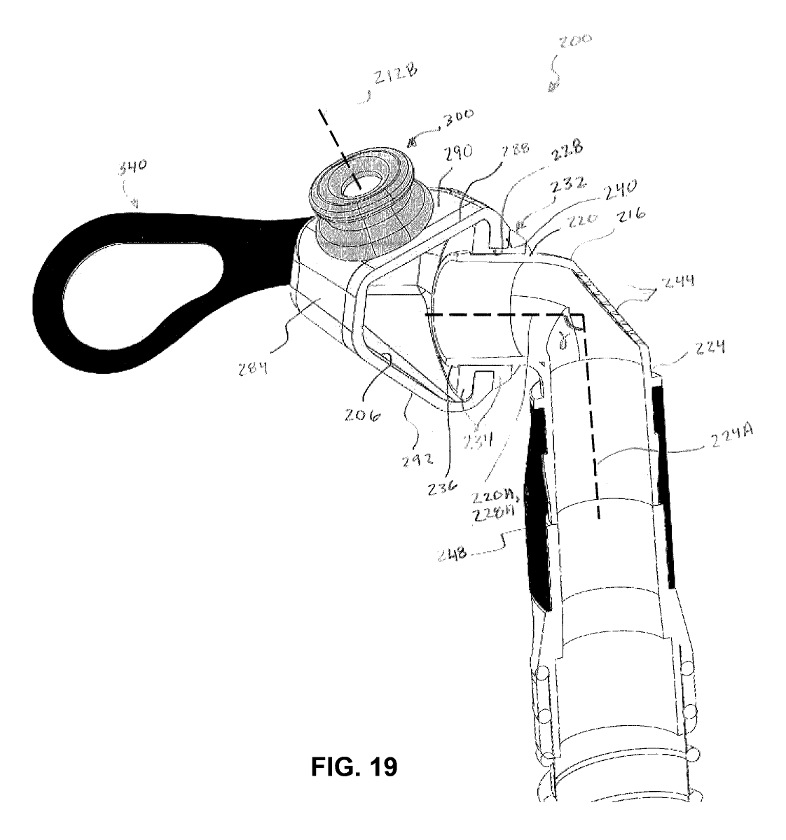

In the illustrated embodiment, an intermediate air tube or connector 216 is positioned between the air supply tube 208 and the interface body 204 to fluidly connect the air supply tube 208 with the cavity 206. The intermediate air tube 216 includes first and second ends 220, 224 with corresponding openings. Referring also to FIG. 19, the intermediate air tube 216 can be angled, bent, or otherwise shaped (e.g., in the form of an elbow) such that central axes 220A, 224A of the openings form an angle γ therebetween ranging from approximately 90 degrees to approximately 120 degrees. The first end 220 of the air tube 216 is configured for removable attachment to the body 204 at the primary opening 228 and is rotatable 360 degrees relative to the body 204 about the central axis 228A.

A sealing member or gasket 232 is removably positioned within the primary opening 228 and includes a pair of flanges 234 that define a groove for receiving the annular wall 240 of the primary opening 228. The first end 220 of the intermediate air tube 216 further includes a tapered annular flange 236 configured to engage the sealing member 232 to retain the intermediate air tube 216 relative to the body 204.

With reference again to FIG. 9, in the illustrated embodiment the air tube 216 includes a plurality of exhalation ports or holes 244. The exhalation holes 244 are defined through the wall of the intermediate air tube 216, between the first and second ends 220, 224. In the illustrated embodiment, the device 200 includes 36 exhalation holes each sized to permit exhalation air to leave the cavity 206 and the intermediate air tube 216, but small enough such that air pressure within is sufficiently maintained. In alternative embodiments, other sizes, numbers, and configurations of exhalation holes 244 may be located anywhere on the intermediate air tube 216, or in another suitable location on the device 200, for example, on the body 204.

With continued reference to FIG. 9, in the illustrated embodiment, the intermediate air tube 216 is rotatably connectable with a snap connection body 248 at the second end 224. In particular, the connection between the air tube 216 and the connection body 248 may be a barbed (somewhat similarly to the connection between the air tube 216 and the sealing member 232) or otherwise connected to permit relative rotation of 360 degrees while resisting relative translation.

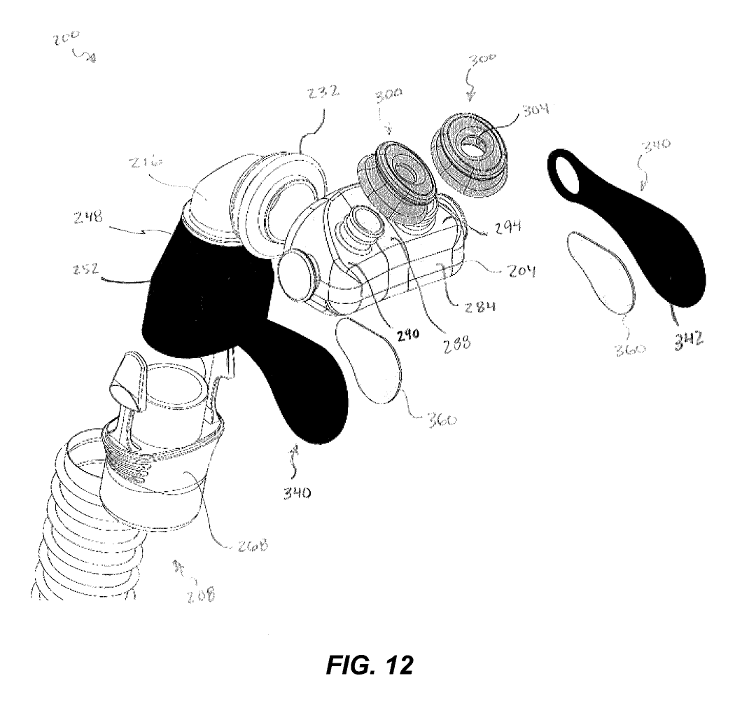

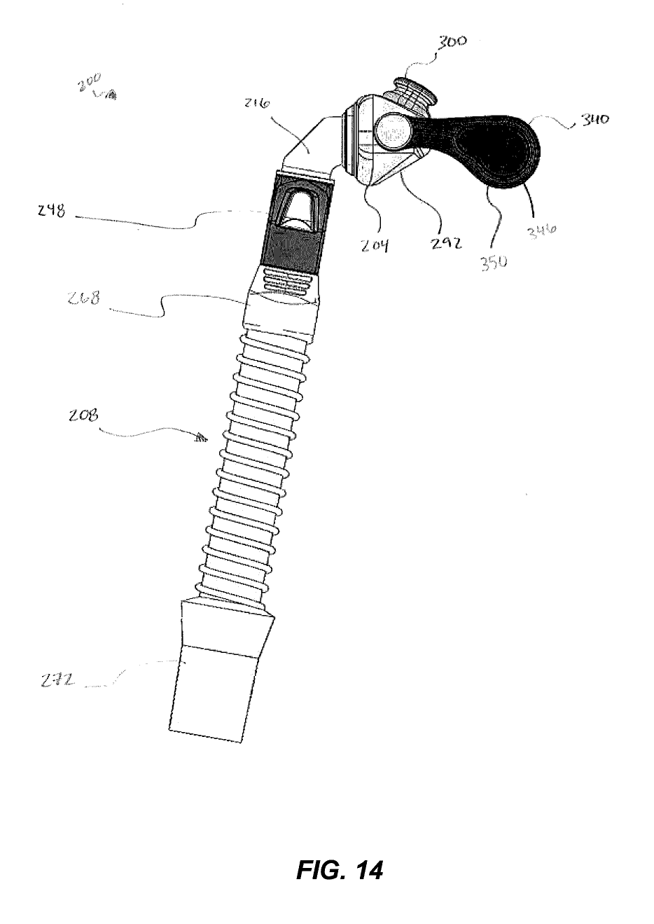

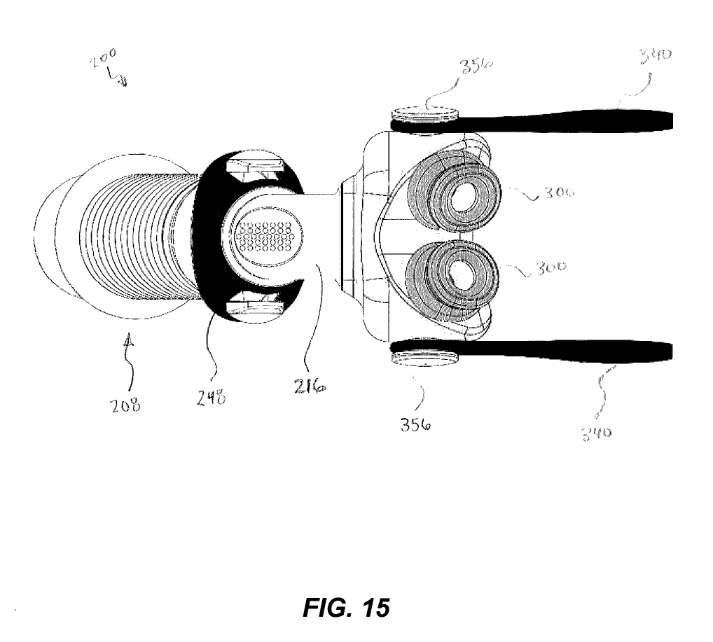

With reference to FIGS. 14-15, in the illustrated embodiment, the connection body 248 has a pair of openings 252, each opening configured to receive a corresponding resilient member 256 extending from a first end 268 of the air supply tube 208. Each of the resilient members 256 includes a radial flange 260, which engages the connection body 248 within the opening 252 to secure the air supply tube 208 to the connection body 248, i.e., in a snap-fit manner. In alternative embodiments, the air supply tube 208 may be removably coupled to the connection body 248 via other mating features (e.g., magnets, threads, connectors, fasteners, etc.). Referring to FIGS. 11 and 12, the air supply tube 208 further includes a second end 272 for connection with a CPAP or other ventilator type device in a known manner, and a corrugated and substantially flexible body 276 between the first and second ends 268, 272.

In operation, the air supply tube 208 is attached to the connection body 248. In particular, each opening 252 of the connection body 248 receives a corresponding one of the resilient members 256 extending from the first end 268 of the air supply tube 208. As the resilient members 256 are inserted, they deflect inwardly until the radial flanges 260 engage the connection body 248 to secure the air supply tube 208 in a snap fit manner.

The connection body 248 is connected to the second end 224 of the intermediate air tube 216. The first end 220 of the intermediate air tube 216 is inserted through the primary opening 228 of the nasal interface body 204, thereby forming a rotatable fluid seal with the sealing member 232. The tapered annular flanges 236 engage the sealing member 232 to retain the intermediate air tube 216 in connection with the nasal interface body 204. Because the intermediate air tube 216 is bent along its length the rotatable connections between the first end 220 and the interface body 204, and the second end 224 and the connection body 248, are in different planes of rotation. In the illustrated embodiment, the planes of rotation are generally orthogonal. However, in alternative embodiments, the intermediate air tube 216 may be bent by any amount for various orientations of the planes of rotation. This allows for a great degree of freedom of movement between the user and the air supply tube 208 without compromising seal integrity.

The central masked area 336 of the nose cover 324 is aligned with the bridge of the user's nose and the nose cover 324 coupled to the surface of a user's nose through application of the contact surface 328, which includes a layer of adhesive. The lack of adhesive on the masked area 336 decreases discomfort caused by adhesive contact with the bridge of the user's nose.

In some embodiments, the nose cover 324 includes a bendable element either embedded within the nose cover 324 or on a surface of the nose cover 324. The bendable element is formed of a material that can be bent to fit the contour of the user's nose and hold its shape, thereby helping the nose cover 324 adhere to the nose. The bendable element may be formed of any suitable material (e.g., a metal such as aluminum). In alternative embodiments, the nose cover 324 includes a tension bow or similar strip in place of the bendable element and formed of a resilient material such that the strip may be deformed when the nose cover 324 is adhered to the exterior of the user's nose. Once the nose cover 324 is adhered to the user's nose the strip applies pressure tending to expand the nasal passageway. Expanding the nasal passageway relieves a tendency of snoring and reduces the air pressure required in the cavity 206. In alternate embodiments, multiple strips may be used to open the nasal passageway along an entire portion of the nasal passageway.

Each of the nasal interface elements 300 is slid over a corresponding one of the nasal interface tubes 212. The material of the nasal interface elements 300 is stretched as the nasal interface elements 300 are slid over the nasal interface tubes 212 providing a holding force to hold the nasal interface elements 300 on the interface tubes 212 and also to form a seal therebetween.

The attachment flaps 340 are each attached to the nasal interface body 204. The ring portion 344 is fitted over the flange 356 and the extension 352 and is retained by the flange portion 356. Once attached, each of the attachment flaps 340 is independently pivotable about the extension 352 relative to the nasal interface body 204.

The nasal interface tubes 212 and corresponding nasal interface elements 300 are inserted into the user's nostrils. Each of the upper radial elements 312 is compressed as the nasal interface elements 300 are inserted. Once inserted the upper radial element 312 expands to make contact with the interior of the nostril at the inner edge of the nostril to hold each of the nasal interface tubes 212 in place. When fully inserted, the lower radial element 316 is compressed against the outer edge of the nostril to provide improved sealing with the nostril of the user. The angled orientation of the nasal interface tubes 212 facilitates increased comfort for the user and improved ergonomic air flow. In addition, the angled orientation of the nasal interface tubes 216 facilitates securing the nasal interface tubes 216 within the nostrils of the user.

The attachment flaps 340 are pivoted up and attached to the nose cover 324 via the hook and loop material (or in another manner) in order to hold the device 200 snug against the nostrils. As such, no straps around the head of the user are required. In alternative embodiments, the nose cover 324 is eliminated and the attachment flaps 340 are adhered directly to the outer surface of the nose of the user.

When connected, the intermediate air tube 216 extends out from the body 204 away from the face of the user, thus keeping the intermediate air tube 216 and the air supply tube 208 from rubbing against the face of the user.

Once the device 200 is assembled air can be supplied to the nostrils of the user via the ventilator device during an inhalation stage. In particular, during the inhalation stage, air is supplied under a positive pressure through the air supply tube 208, through the intermediate air tube 216 and into the cavity 206. The air enters the cavity 206 through the primary opening 228 and is directed toward the nasal interface tubes 212 by the second wall 288 and the third wall 292 into the user's nostrils (i.e., air is deflected upward toward the nasal interface tubes 212 by the third wall 292 and funneled downward by the second wall 288 toward the nasal interface tubes 212). The sidewalls 296 are also contoured to direct the air toward and through the nasal interface tubes 212. The air travels through the nasal interface tubes 212 into the user's nostrils where it is then inhaled by the user.

During an exhalation stage, exhalation air is exhaled through the user's nostrils and exits through the nasal interface tubes 212 and into the cavity 206. The exhalation air flows from the cavity 206 through the primary opening 228 into the intermediate air tube 216, where the exhalation air then exits the device 200 through the exhalation holes 244.

While the above describes example embodiments of the present disclosure, these descriptions should not be viewed in a limiting sense. Rather, several variations and modifications can be made without departing from the scope of the present disclosure.

What is claimed is:

1. A breathing interface device comprising:
a housing defining a chamber, the housing including
a first opening in fluid communication with the chamber and a positive pressure source,
a first nostril tube in fluid communication with the chamber and having a first central axis, and
a second nostril tube in fluid communication with the chamber and having a second central axis, the first nostril tube and the second nostril tube configured such that the first central axis of the first nostril tube and the second central axis of the second nostril tube intersect to form an angle; and
a nasal interface element removably couplable to the first nostril tube, the nasal interface element compressible to fit within a nostril of a user and expandable to make contact with an inner edge of the nostril.

2. The device of claim 1, wherein the angle is greater than about 20 degrees.

3. The device of claim 1, wherein the angle is between about 20 degrees and about 40 degrees.

4. The device of claim 1, wherein the angle is about 27 degrees.

5. The device of claim 1, wherein the housing includes a wall having a first portion and second portion, wherein the first nostril tube extends from the first portion away from the chamber and the second nostril tube extends from the second portion away from the chamber, wherein the central longitudinal axis of the first nostril tube is orthogonal to the first portion and the central longitudinal axis of the second nostril tube is orthogonal to the second portion.

6. The device of claim 1, wherein one of the first and second nostril tubes includes an annular rib on an outer surface thereof.

7. The device of claim 1, further comprising a facial patch removably couplable to a portion of the user's face, the facial patch having a contact surface, wherein a portion of the contact surface includes an adhesive.

8. The device of claim 7, further comprising at least one attachment flap pivotally couplable to the housing and removably attachable to the facial patch.

9. The device of claim 1, further comprising a connector removably attachable to the housing at a first opening and attachable to a positive pressure source at a second opening, the connector further including a plurality of openings in a connector wall.

10. The device of claim 1, further comprising a second nasal interface element removably couplable to the second nostril tube, the second nasal interface element compressible to fit within a nostril of a user and expandable to make contact with an inner edge of the nostril.

11. A breathing interface device comprising:
a housing defining a chamber, the housing including
a first nostril tube in fluid communication with the chamber,
a second nostril tube in fluid communication with the chamber, and
a first opening in fluid communication with the chamber and a positive pressure source;
a facial patch adherable to a portion of a user's face and having a contact surface;
an attachment flap rotatably couplable to the housing and releasably couplable to the facial patch; and
a nasal interface element removably couplable to the first nostril tube, the nasal interface element compressible to fit within a nostril of a user and expandable to make contact with an inner edge of the nostril.

12. The device of claim 11, wherein the contact surface of the facial patch includes a masked area free of adhesive.

13. The device of claim 12, wherein the facial patch at least partially covers a user's nose, and wherein the masked area extends along a centerline of the facial patch.

14. The device of claim 12, wherein the masked area has a width between 1.0 cm and 2.0 cm.

15. The device of claim 11, wherein the facial patch includes a surface presenting one element of hook and loop attachment material and the attachment flap includes a surface presenting the other element of hook and loop attachment material.

16. The device of claim 11, wherein the attachment flap includes a ring portion that receives an extension extending from the housing.

17. The device of claim 11, wherein the first nostril tube has a first central axis and the second nostril tube has a second central axis, and wherein the first nostril tube and the second nostril tube are configured such that the first central axis of the first nostril tube and the second central axis of the second nostril tube intersect to form an angle.

18. A breathing interface device comprising:
a housing defining a chamber, the housing including
a first opening in fluid communication with the chamber and a positive pressure source, the first opening having a central opening axis,
a first nostril tube in fluid communication with the chamber,
a second nostril tube in fluid communication with the chamber, and
a wall having a surface angled relative to the central opening axis of the first opening, the first nostril tube extending from the surface; and
a nasal interface element removably couplable to the first nostril tube, the nasal interface element compressible to fit within a nostril of a user and expandable to make contact with an inner edge of the nostril.

19. The device of claim 18, wherein the first nostril tube has a first central tube axis and the second nostril tube has a second central tube axis, and wherein the first nostril tube and the second nostril tube are configured such that the first central tube axis of the first nostril tube and the second central tube axis of the second nostril tube intersect to form an angle.

20. The device of claim 18, wherein the angle is greater than about 20 degrees.
